(19) United States
(12) Patent Application Publication
(10) Pub. No.: US 2007/0128075 A1
(43) Pub. Date: Jun. 7, 2007

(54) ELECTRICAL CONTACT DEVICE FOR TEST SPECIMEN

(76) Inventor: Gunther BÖHM, Nufringen (DE)

Correspondence Address: OSTROLENK FABER GERB & SOFFEN, 1180 AVENUE OF THE AMERICAS, NEW YORK, NY, 100368403, US

(21) Appl. No.: 11/566,908

(22) Filed: Dec. 5, 2006

(30) Foreign Application Priority Data
Dec. 5, 2005 (DE) 10 2005 058 762.3
Nov. 21, 2006 (DE) 10 2006 054 735.7

(51) Int. Cl. G01N 33/48

(52) U.S. Cl. 422/68.1

(57) ABSTRACT

An electrical contact device for an electrical test specimen, comprising test contacts for making electrical touching contact with the test specimen and a supporting apparatus assigned to an intermediate component for holding the contact device in a test machine or prober. In an embodiment of a permanent modular unit, the supporting apparatus is connected to a dedicated intermediate component associated only with the respective contact device. An electrical test apparatus comprises an electrical contact device.

### BACKGROUND OF THE INVENTION

The invention relates to an electrical contact device for an electrical test specimen, comprising test contacts for making electrical touching contact with the test specimen and comprising a supporting apparatus that can be assigned an intermediate component for holding the contact device in a test machine (prober).

Electrical contact devices of the type mentioned in the introduction serve for electrically testing an electrical test specimen, for example a wafer. For the electrical testing, the contact device is inserted into a test machine. The contact device has a multiplicity of test contacts embodied as buckling needles, for example. The free ends of the buckling needles serve for making touching contact with the test specimen. The test machine has the task of positioning the test specimen beneath the contact device (X and Y alignment) and raising the test specimen (Z alignment) in such a way that the buckling needles make touching contact with corresponding contacts of the test specimen for the test. Furthermore, the test machine has the task of producing electrical connections between the contact device and a tester. The electrical testing of the test specimen is carried out by means of the tester, that is electrical test circuits are established towards the test specimen in order to carry out a functional test. The test current paths run from the tester via the contact device to the test specimen, and from there back to the tester. Cameras are preferably employed for the alignment of contact device and test specimen, which cameras detect the position of the test specimen on a so-called test specimen carrier (chuck) of the test machine (prober) and the position of the needle tips of the buckling needles very precisely (to an accuracy of a few μm) and thereby enable a sufficiently accurate alignment of the components with respect to one another, so that, in the course of contact-making, the buckling needles acquire contact precisely with the test specimen contacts. Many contact-making operations are usually required in order to test a test specimen, for example a wafer, comprehensively with regard to functionality. Entirely satisfactory testing necessitates forming contact-reliable test current paths between the tester and the test specimen. This necessitates positioning the test specimen very precisely beneath the contact device and then pressing it onto the test contacts of the contact device with suitable press-on force by means of a Z upward movement. A supporting apparatus (stiffener) has the task of diverting the forces that occur as a result of the test specimen being pressed onto the contact device to the test machine reliably and without the occurrence of impermissible warpages and/or flexures. One known test machine has an intermediate component embodied as an intermediate ring (probecard holder ring) for this purpose. The intermediate ring, assigned to the test machine, is mounted such that it can be moved in the test machine. The test machine has at the front a flap which opens if the intermediate ring moves out like a kind of drawer, so that it can be equipped with the contact device. The arrangement described above corresponds approximately to the principle of a CD-ROM drive that opens by virtue of a drawer moving out in order to introduce the CD. For the aforementioned equipping, the contact device is introduced into the intermediate ring and screwed to the latter by means of a plurality of small threaded screws distributed on the circumference. In this case, centering pins in the intermediate ring and corresponding holes in the contact device enable accurate alignment of the contact device with respect to the intermediate ring. The test machine can then accommodate the intermediate ring together with contact device in its interior by inward movement, so that the contact device is displaced to the location provided for the test. Rotation and/or raising may also be effected in this case. Finally, the intermediate ring is locked in the test machine. An intermediate ring of this type is also provided in the case of a test machine without drawer loading and unloading, which intermediate ring is part of the test machine and is always positioned in it or is configured such that it is insertable into said test machine.

In the case of relatively large contact devices (for example vertical test cards) having a multiplicity of test contacts, currently more than a thousand test contacts (test needles), preferably vertical forces arise in the contact device in the course of the contact-making. Depending on the number of test contacts (test needles), test forces of for example between 10 N and 4000 N may occur, which are taken up by the abovementioned supporting apparatus (stiffener) and are passed onto the intermediate ring and transmitted by the latter to the test machine. In order to ensure an entirely satisfactory function of the overall system, it is necessary to construct the components contact device and intermediate ring with very high torsional stiffness since the test contacts associated with the contact device, particularly if they are embodied as buckling needles or buckling wires, only have a very small maximum contact travel (for example 50 μm up to 200 μm) and, consequently, during contact-making, a torsion and/or flexure of the overall system must be very small relative to said maximum contact travel. By way of example, if the test machine wishes to set a contact travel of 150 μm and the system exhibits inadequate stiffness, so that, due to flexing, for example, a travel contact that takes effect effectively at the respective test contacts is significantly smaller, for example only 70 μm, it is not ensured that contact will be made with the test specimen reliably.

### SUMMARY OF THE INVENTION

Therefore, the invention is based on the object of specifying an electrical contact device of the type mentioned in the introduction which enables contact to be made with a test specimen reliably and reproduceably, for the purpose of testing said test specimen.

This object is achieved according to the invention by virtue of the fact that for the embodiment of a permanent modular unit, the supporting apparatus is connected to a dedicated intermediate component associated only with this contact device. Accordingly, the test machine is no longer assigned a single intermediate component, in particular an intermediate ring, into which the various contact devices are insertable. Instead each contact device has a dedicated intermediate component. Irrespective of whether or not the contact device is inserted into the test machine, it always has its dedicated intermediate component which is optimally connected to the supporting apparatus on account of individual adaptation and an interface problem that might become apparent as inadequate stiffness of the system, for example, therefore does not occur. The aforementioned interface now does not have to be produced upon each occasion when a contact device is inserted into the test machine. Instead, it exists permanently between the supporting apparatus and the intermediate component since the intermediate component is always assigned to the associated contact device. Therefore, there is no need to take account of an interface that always has to be resolved and then reestablished, and a significantly stiffer composite can be realized according to the invention.

According to one development of the invention it is provided that the supporting apparatus and the intermediate component are connected to one another to form a common component. This connection does not have to be interface-free, that is the two parts can be screwed to one another for their connection, for example. But, this screwed connection is not permanently opened and reestablished. Instead, these components are connected during the production of the contact device and then remain connected, with the result that high accuracy and precision are present. The aforementioned common component may also be produced, in particular, by adhesive bonding of supporting apparatus and intermediate component, whereby a highly loadable and precise connection can likewise be produced.

According to one development of the invention, the supporting apparatus and the intermediate component are integrally connected to one another. The aforementioned interface is even obviated as a result of this, so that a very stiff and highly accurate construction is present.

According to one development of the invention, the contact device is embodied as a test card. The test card is preferably a vertical test card. This means that it has test contacts, in particular test pins or test needles, preferably buckling needles or buckling wires, that are oriented vertically with respect to a horizontal position. The test contacts are preferably associated with a contact head. The test contacts of the contact head are used to make electrical touching contact with the test specimen during the testing thereof.

According to one development of the invention, the supporting apparatus has a front support and a rear support. The front support is situated on that side of the contact device which faces the test specimen. Accordingly, the rear support lies on that side of the contact device which lies remote from the test specimen.

In particular, it is provided that the rear support is connected to the intermediate component for the embodiment of the permanent modular unit. The front support is preferably connected to the rear support.

Preferably, a wiring carrier, in particular a printed circuit board, is arranged between the front support and the rear support. The printed circuit board has contacts on its side assigned to the contact head, which contacts are connected via corresponding conductor tracks to contacts that are situated on its rear side, that is on the side remote from the test head. Whereas the contacts on the front side of the wiring carrier lie very close together and are made very small in order that contact can be made with the contacts of the test specimen, which lie correspondingly close together, via the interposed test contacts, the contacts arranged on the rear side of the wiring carrier are embodied such that they are larger and further apart from one another, so that the tester already mentioned can be connected without any problems. Accordingly, the wiring carrier serves as a conversion device for converting a very narrow contact spacing to a larger contact spacing.

It is furthermore advantageous if contacts of the wiring carrier are in touching contact with the test contacts of the contact head. This has already been discussed above. The test contacts are therefore not fixedly connected to the contacts of the wiring carrier, but rather by the bearing of the preferably vertically extending buckling needles or buckling wires on the contacts of the wiring carrier. Said bearing is supported by the contact pressure in the course of making contact with the test specimen.

The invention furthermore relates to an electrical test apparatus for the testing of an electrical test specimen, comprising a test machine (prober), into which a contact device serving for making touching contact with the test specimen, in particular a contact device as described above, selectable from a multiplicity of preferably different contact devices, is insertable in each case. The apparatus further comprises at least one intermediate component for holding the contact device respectively used in the test machine, each contact device being equipped with a dedicated intermediate component for the respective embodiment of a permanent modular unit. If a contact device is thus inserted into a test machine of this type, then the contact device has its dedicated intermediate component, in particular its dedicated intermediate ring, which always remains at the contact device, irrespective of whether the latter is currently being used for testing and is thus situated in the test machine, or is stored outside the test machine for later deployment purposes. Accordingly—unlike in the prior art, it is no longer the case that a contact device is respectively assigned one and the same intermediate component which is associated with the test machine and is accordingly present only once, so that different contact devices which serve for testing different test specimens always have to be connected to the same intermediate component. This is remedied by the invention in that the number of intermediate components present is the same as the number of contact devices, that is each contact device has its dedicated intermediate component, so that each contact device together with the intermediate component forms a permanent modular unit.

### BRIEF DESCRIPTION OF THE DRAWINGS

The drawings illustrate the invention on a basis of exemplary embodiments, to be precise:

FIG. 1 shows a cross section through an electrical test apparatus for the testing of an electrical test specimen,

FIG. 2 shows a cross section through a contact device inserted into a test machine of the test apparatus and serving for making contact with the test specimen, and

FIG. 3 shows a further exemplary embodiment of a contact device

### DESCRIPTION OF PREFERRED EMBODIMENTS

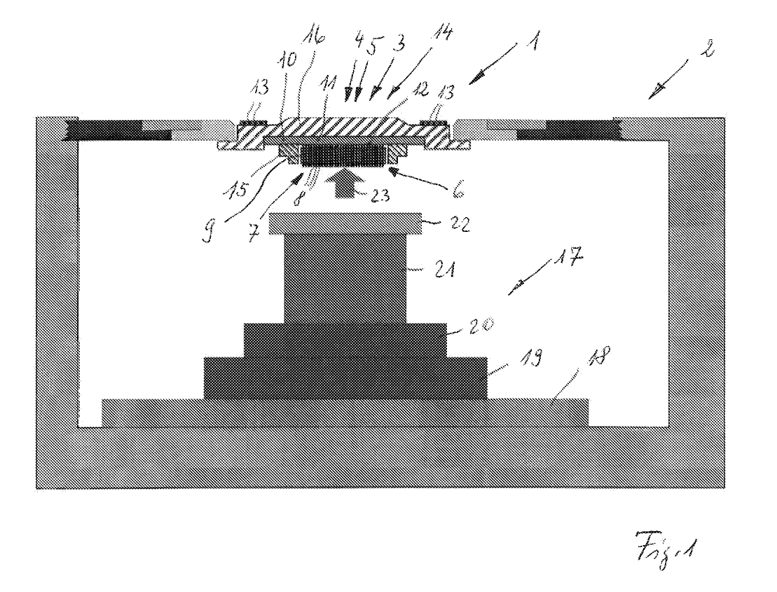

FIG. 1 shows an electrical test apparatus 1 serving for testing a test specimen, which is not revealed in FIG. 1. The test apparatus 1 has a test machine 2 (prober), into which a contact device 3 is inserted. The contact device 3 is preferably inserted into the test machine 2 by means of a drawer construction similar to that of a CD-ROM drive. The contact device 3 is embodied as a test card 4, in particular a vertical test card 5. This last means that it has a multiplicity of test contacts 7 in a contact head 6, said test contacts being embodied as needles, in particular buckling needles 8, which run transversely, in particular vertically, with respect to the preferably horizontal test plane. “Buckling needles” means that they have in each case a slight flexure, that is they deviate from a rectilinear form. The flexure may be brought about for example by holding openings of a guide 9 which lie in offset fashion and in which the buckling needles are mounted in longitudinally displaceable fashion. If the test specimen is pressed against the free ends, of the buckling needles 8, which preferably run to a point, then the latter can spring out slightly on account of the flexure and thereby compensate for spacing irregularities and establish very good contact.

As already mentioned above, the buckling needles 8 are held in the guide 9, one of the ends of the buckling needles 8 respectively forming free ends serving for making touching contact with the test specimen. The other ends of the buckling needles 8 bear on contacts 12 of a wiring carrier 10, preferably of a printed circuit board 11, of the contact arrangement. The aforementioned contacts 12 of the printed circuit board 11 are connected to contacts 13 lying on the other side of the printed circuit board 11, for example via conductor tracks 41 of the printed circuit board 11. The contacts 13 are connected to a tester, which is not shown in FIG. 1 and which serves for connecting test current paths through to the test specimen in order to test the test specimen with regard to electrical functionality. Whereas the contacts 12 of the printed circuit board 11 which are touched by the buckling needles 8 lie extremely close together, the contacts 13 can be arranged in a manner distributed over a much larger area, so that the tester can be connected without any problems. A support device 14, comprising a front support 15 and a rear support 16, is provided for stiffening the contact device 3. The support device 14 serves to take up the contact pressure that arises when the test specimen, as described in more detail below, is pressed against the buckling needles 8 for the purpose of making touching contact.

The test machine 2 has a test specimen carrier 17 (chuck) having a stationary baseplate 18. Furthermore, the test specimen carrier 17 includes a Y positioning device 19, an X positioning device 20 and a Z positioning device 21. Arranged on the Z positioning device 21 is a vacuum mount 22, by means of which the test specimen can be held in a positionally invariable manner with respect to the vacuum mount 22 by vacuum. If the test specimen, for example a wafer, is then placed onto the vacuum mount 22 in planar fashion and held by vacuum, then it can be positioned beneath the contact device 3 in a positionally accurate manner by means of the X and Y positioning devices 20 and 19 and with the aid of cameras in such a way that in the course of making touching contact, the buckling needles 8 make contact with corresponding contacts of the test specimen in a positionally accurate manner. For the contact making, the Z positioning device 21 moves upward and presses the test specimen against the free ends of the buckling needles 8. This movement is indicated by means of an arrow 23 in FIG. 1.

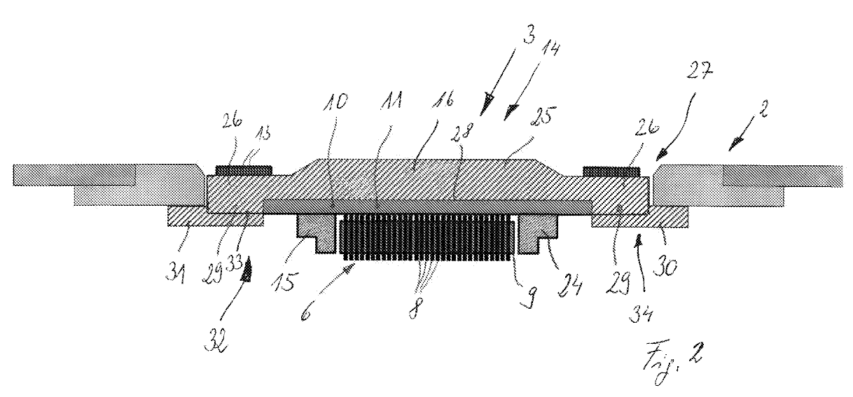

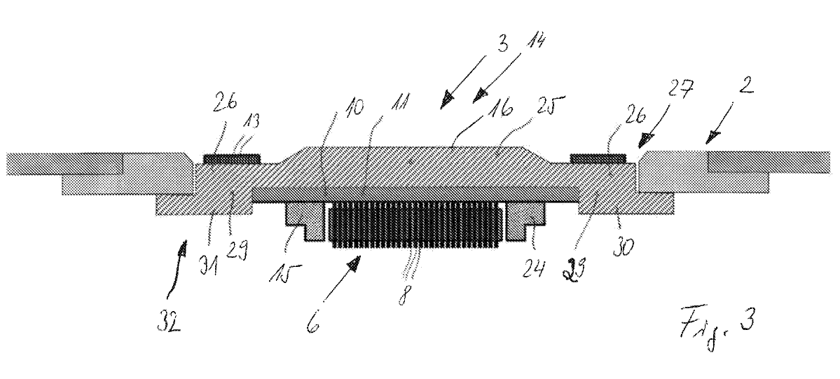

FIG. 2 shows the electrical contact device 3 from FIG. 1 in an enlarged illustration. This is a cross-sectional view. It can be assumed in principle that the wiring carrier 10 is embodied as a planar component, in particular as a circular disk. This correspondingly holds true for the contact head 6, which preferably likewise has a form like a circular disk. Of course, other forms of the contact head such as, for example, rectangular or square disk-like forms are also conceivable. When viewed three-dimensionally, the supporting apparatus 14 is likewise embodied in planar fashion. In particular, the front support 15 forms a stiffening ring 24 and the rear support 16 forms a stiffening plate 25, from which proceed a multiplicity of supporting arms 26, distributed over the circumference. Preferably, eight supporting arms 26 are provided in a manner distributed over the circumference. It is also possible to provide more or fewer supporting arms 26. The test machine 2 has a receptacle 27 for receiving the contact device 3. The supporting arms 26 project beyond a supporting area 28 of the rear support 16 with supporting arm sections 29 which lie in edge-open slots of the wiring carrier 10. The edge-open slots cannot be seen in FIG. 2 on account of the chosen section effected for the cross-sectional illustration. The supporting arms 26 are connected to an intermediate component 30 in the region of their supporting arm section 29, which intermediate component is embodied as an intermediate ring 31 and can be fixed/locked in a positionally accurate manner in the test machine 2. Corresponding coupling means are provided for this purpose, but they are not illustrated in FIG. 2. What is of importance, then, is that the supporting apparatus 14, as can be seen from FIG. 2, is connected to the intermediate component 30, in such a way that a permanent modular unit 32 is formed, that is the contact device 3 has a dedicated intermediate component 30 associated with it. The intermediate component 30 is accordingly not associated with the test machine 2, rather each contact device 3 inserted into the test machine 2 has a dedicated intermediate component 30 in fixedly connected fashion, so that when the contact device 3 is inserted into the test machine 2, it is not necessary for a mechanical interface of the abovementioned type to be closed, rather these components are already fixedly connected to one another. This may be a fixed solid screwed connection, an adhesively bonded connection 33 as indicated in FIG. 2, or else an integral embodiment, revealed in FIG. 3. Thus while in FIG. 2 the supporting apparatus 14 is connected to the intermediate component 30 in order to form a common component 34, for example by means of the aforementioned adhesively bonded connection 33, the rear support 16 being connected to the intermediate component 30 in the case illustrated in FIG. 2, the exemplary embodiment of FIG. 3 manifests an integral nature, that is the rear support 16 and the intermediate component 30, which is embodied in particular as an intermediate ring 31, are integrally connected to one another. In the latter case, an interface is completely dispensed with, with the result that overall the construction is particularly stiff, with the result that no instances of torsion or deformation which adversely influence the contact-making occur in the course of making contact with the test specimen. The front support 15 is connected to the rear support 16 via connecting means (not illustrated) reaching through the wiring carrier 10.

1. An electrical contact device for an electrical test specimen, comprising
a plurality of test contacts extruding in a direction such that each contact makes electrical touching contact with the test specimen,
a supporting apparatus having an intermediate component operable for holding the contact device in a test machine (prober),
the supporting apparatus is connected to a dedicated one of the intermediate components and that component is associated only with the contact device.

2. The contact device according to claim 1, wherein the supporting apparatus and the intermediate component are connected to forming a common component.

3. The contact device according to claim 1, wherein the supporting apparatus and the intermediate component are integrally connected to one another.

4. The contact device according to claim 1, further comprising a test card on which the test contacts are disposed.

5. The contact device according to claim 4, wherein the test card is embodied as a vertical test card.

6. The contact device according to claim 1, further comprising a contact head for supporting the test contacts for making electrical touching contact.

7. The contact device according to claim 6, wherein the contact head has test contacts embodied as needles.

8. The contact device according to claim 1, wherein the supporting apparatus has a front support and a rear support.

9. The contact device according to claim 8, wherein the rear support is connected to the intermediate component for embodying the permanent modular unit.

10. The contact device according to claim 8, further comprising a wiring carrier arranged between the front support and the rear support.

11. The contact device according to claim 10, further comprising contacts of the wiring carrier are in touching contact with the test contacts of the contact head.

12. An electrical test apparatus for the testing of an electrical test specimen, comprising
a test machine (prober),
a contact device according to claim 1 disposed in the test machine for making touching contact with the test specimen, wherein the contact device is selectable from a multiplicity of different ones of the contact devices, at least one intermediate component for holding the selected contact device respectively used in the test machine, and each contact device is equipped with a dedicated intermediate component for a respective embodiment of a permanent modular unit of the intermediate component and the contact device.

13. The contact device according to claim 7, wherein the needles are buckling needles.

14. The contact device of claim 10, wherein the wiring carrier comprises a printed circuit board.
